(19) United States
(12) Patent Application Publication
(10) Pub. No.: US 2023/0190601 A1
(43) Pub. Date: Jun. 22, 2023

(54) HAIR CONDITIONING COMPOSITION

(71) Applicant: The Procter & Gamble Company, Cincinnati, OH (US)

(72) Inventors: Shikhar GUPTA, Singapore (SG); Nobuaki UEHARA, Singapore (SG)

(21) Appl. No.: 18/079,023

(22) Filed: Dec. 12, 2022

Related U.S. Application Data
(60) Provisional application No. 63/290,070, filed on Dec. 16, 2021.

(51) Int. Cl. A61K 8/36; A61Q 5/12; A61K 8/06

(52) U.S. Cl. CPC A61K 8/361; A61Q 5/12; A61K 8/064

(57) ABSTRACT

A hair conditioner composition comprising a first material and a second material; wherein the first material comprises at least two amine groups, has an A Log P at pH 6 of less than about −2, and has a hydrophobic surface area at pH 6 of at least about 160 Å² (Angstrom)²; and wherein the second material comprises at least one C12 to C22 carbon chain and has an A Log P at pH 6 of at least about 3.2.

### FIELD OF THE INVENTION

The present invention relates to a hair conditioning composition comprising a first material and a second material, all of which form a gel structure in formulation.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds and an aqueous carrier, are believed to provide a gel structure, which is suitable for providing a variety of conditioning benefits such as a slippery feel during the application to wet hair, softness, and a moisturized feel on dry hair.

But some consumers would prefer products that do not have cationic surfactant molecules. Thus, there is a continuing need to find alternative surfactants, particularly green chemistry surfactants that can still form a gel structure in formulation and deliver the consumer-desired benefits.

None of the existing art provides all of the advantages and benefits of the present invention, including performance, cost, safety, sustainable sourcing, and being environmental-friendly.

### SUMMARY OF THE INVENTION

The present invention is directed to A hair conditioner composition comprising:
a first material and a second material;

wherein the first material comprises at least two amine groups, has an A Log P at pH 6 of less than about −2, and has a hydrophobic surface area at pH 6 of at least about 160 Å² (Angstrom)²; and

wherein the second material comprises at least one C12 to C22 carbon chain and has an A Log P at pH 6 of at least about 3.2.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Q.S. herein means up to 100%.

### Composition

The hair conditioning composition of the present invention comprising:

a) a first material and a second material;

wherein the first material comprises at least two amine groups, has an A Log P at pH 6 of at less than about −2, and has a hydrophobic surface area at pH 6 of at least about 160 Å² (Angstrom)²; and
wherein the second material comprises at least one C12 to C22 carbon chain and has an A Log P at pH 6 of at least about 3.2;

b) a high melting point fatty alcohol; and

c) an aqueous carrier;
wherein a) through c) form a gel structure.

The objective of the invention is to provide stable conditioner compositions containing a gel structure and having a thick yet melting application feel, clean rinse, and dry look and conditioning benefits. The presence of a material comprising a first material and a second material, wherein the first material has at least two amine groups, has an A Log P of at less than about −2, and has a hydrophobic surface area at pH 6 of at least about 160 Å² (Angstrom)², while the second material comprises at least one C12 to C22 carbon chain and has an A Log P at pH 6 of at least about 3.2, along with a fatty alcohol and an aqueous carrier, leads to conditioner compositions that have are stable, have acceptable rheology, and also provide consumer-expected conditioning benefits, such as hair manageability, frizz control, and volume.

A typical hair conditioner composition comprises a structurant, such as a fatty alcohol, a feel/rheology modifier polymer, a conditioning oil and agent, preservation ingredients, perfumes/colorants, plus a cationic surfactant. As consumers become more interested in products comprising only natural and gentle ingredients, each of these hair conditioner components is examined for how consumer-perceived natural and gentle ingredients can make up the composition, while still providing the performance and benefits that consumers expect. One area that may be modified is the surfactant. Rather than use a cationic surfactant molecule, the present inventors have formulated hair conditioner compositions comprising a material comprising a first material and a second material wherein the first material has at least two amine groups, has an A Log P of at less than about −2, and has a hydrophobic surface area at pH 6 of at least about 160 Å² (Angstrom)², and wherein the second material comprises at least one C12 to C22 carbon chain and has an A Log P at pH 6 of at least about 3.2. The conditioner may also comprise a high melting point fatty alcohol, and an aqueous carrier. Integrating various materials with the described first and second materials allows flexibility in molecular design and the supply chain, all with natural and gentle ingredients, while still meeting performance, cost, safety, sustainable sourcing and environmental-friendly criteria.

### Gel Structure

The compositions of the present invention comprise a gel structure or gel network, in some cases a lamellar gel network matrix with a L_{β} phase. The gel structure is suitable for providing various conditioning benefits, such as slippery feel during the application to wet hair and softness and moisturized feel on dry hair.

A gel structure in a conditioner composition is demonstrated by the lack of phase separation of the composition. When the first material (a-1), with its negative A log P, is combined with the second material (a-2), with a more positive A log P, the combination forms the gel structure together with (b) high melting point of fatty alcohol and (c) water. If the combination is not balanced, then the conditioner composition can exhibit phase separation, which is not desirable to consumers.

In some embodiments, the second material may be, but is not limited to, a fatty acid, sugar esters, glycerol esters and ethers with at least one double bond, and sulphates. In some embodiments, the second material may be, but is not limited to, sucrose distearate (SDE), behenic acid, stearic acid, oleic acid, stearyl fumarate, glyceryl monostearate, linoleic acid, palmitic acid, stearoyl lactylate, glyceryl monooleate (GMO), sucrose dilaurate (SDL), ricinoleic acid, stearyl sulfate, lauric acid, sucrose dipalmitate, glyceryl palmitoleate, glyceryl lionleate, and glyceryl linolenate. In some embodiments, the second material may be selected from the materials listed in Table 4 that have an A log P at pH 6 of at least about 3.2. Table 4 also includes materials that do not have an A log P at pH 6 of at least 3.2 and that can exhibit phase separation when formulated into a hair conditioner composition, as shown in Comparative Examples.

TABLE 4

| Second material | ALogP at pH 6 |
|---|---|
| sucrose distearate (SDE) | 11.467 |
| Behenic Acid | 8.042 |
| Stearic acid | 6.565 |
| oleic acid | 6.48 |
| stearyl fumarate | 6.224 |
| Glyceryl Mono stearate | 6.217 |
| LinoleicAcid | 6.036 |
| Palmitic_Acid | 5.993 |
| stearoyl lactylate | 5.773 |
| Glyceryl Monooleate | 5.328 |
| (GMO) | |
| Sucrose dilaurate (SDL) | 5.305 |
| RicinoleicAcid | 4.905 |
| Stearyl sulfate | 4.536 |
| Lauric Acid | 3.48 |
| Dodecyl glucoside | 2.873 |
| Sucrose monolaurate | 0.841 |

In some embodiments, the first material may be, but is not limited to, canavanine, lysine, ornithine, arginine, citrulline, methylarginine, laminine, ethanimidoylornithine, n-dimethylarginine, norleucine, argininamide, DMAPA, propyl arginine, histidine, and 4-aminophenylalanine. In some embodiments, the first material may be, but is not limited to, canavanine or ornithine. In some embodiments, the first material may be selected from the materials listed in Table 5 that have an A log P at pH 6 of less than −2. The table also includes materials that do not have an A log P of less than −2 and that can exhibit phase separation when formulated into a hair conditioner composition, as shown in Comparative Examples.

The hair conditioner composition may comprise from about 0.01% to about 15%, by weight of the composition, of the first material, preferably from about 0.05% to about 10%, more preferably from about 0.1% to about 5%, by weight of the hair conditioner composition. The hair conditioner composition may comprise from about 0.01% to about 15%, by weight of the composition, of the second material, preferably from about 0.05% to about 10%, more preferably from about 0.1% to about 5%, by weight of the hair conditioner composition.

The following Table 5 lists possible first materials, along with each A Log P at a pH of 6 and hydrophobic surface area at pH 6, in Angstroms².

TABLE 5

| | | Hydrophobic |
|---|---|---|
| | ALogP@ | Surface Area @ |
| First material | pH 6 | pH 6 |
| Canavanine | −4.80 | 179 |
| Lysine | −4.70 | 175 |
| Ornithine | −4.70 | 176 |
| Arginine | −3.93 | 198 |
| Citrulline | −3.89 | 195 |
| Methylarginine | −3.73 | 239 |
| Laminine | −3.63 | 293 |
| Ethanimidoylornithine | −3.62 | 229 |
| n-dimethylarginine | −3.52 | 269 |
| norleucine | −3.22 | 186 |
| argininamide | −3.09 | 210 |
| DMAPA | −3.01 | 238 |
| (Dimethylaminopropylamine) | | |
| propyl arginine | −2.86 | 292 |
| Histidine | −2.58 | 181 |
| 4-aminophenylalanine | −2.35 | 224 |
| Guanidine | −1.09 | 54 |
| Urea | −1.04 | 57 |
| Hydroxyurea | −1.03 | 79 |
| Guanine | −0.66 | 153 |

In some embodiments, the surfactant may further comprise cationic surfactants. Suitable cationic surfactants may include, for example, behentrimonium methosulfate (BTMS), behentrimonium chloride (BTMAC), stearamidopropyldimethylamine (SAPDMA), behenamidopropyldimethylamine (BAPDMA), brassicyl valinate esylate, and combinations thereof.

The hair conditioner compositions may comprise at least about 60% of an aqueous carrier, by weight of said hair conditioner composition, and in some embodiments at least about 80%.

In general, the mixture of the materials described herein, along with an aqueous carrier, may have a pH of at least about 4.5. The ratio of the first material to the second material may be from about 1:40 to about 40:1, preferably from about 1:15 to about 30:1, or more preferably from about 1:10 to about 20:1.

The compositions of the present invention may be substantively free of ceramide. The compositions of the present invention may be substantively free of cholesterol. And the compositions of the present invention may be substantively free of a gel network made of only non-ionic surfactant.

### High Melting Fatty Alcohol

The high melting point fatty alcohol can be included in the composition at a level of from about 2%, preferably from about 4%, more preferably from about 5%, still more preferably from about 5.5%, and to about 15%, preferably to about 10% by weight of the composition, in view of providing the benefits of the present invention.

The high melting point fatty alcohol useful herein have a melting point of 25° C. or higher, preferably 40° C. or higher, more preferably 45° C. or higher, still more preferably 50° C. or higher, in view of stability of the lamellar gel network matrix with Lβ. Preferably, such melting point is up to about 90° C., more preferably up to about 80° C., still more preferably up to about 70° C., even more preferably up to about 65° C., in view of easier manufacturing and easier emulsification. In the present invention, the high melting point fatty alcohol can be used as a single alcohol or as a blend or mixture of at least two high melting point fatty alcohols. When used as such blend or mixture, the above melting point means the melting point of the blend or mixture.

The high melting point fatty alcohol useful herein is selected from the group consisting of fatty alcohols, fatty alcohol derivatives, fatty acid derivatives, and mixtures thereof. It is understood by the artisan that the alcohols disclosed in this section of the specification can in some instances fall into more than one classification, e.g., some fatty alcohol derivatives can also be classified as fatty acid derivatives. However, a given classification is not intended to be a limitation on that particular alcohol but is done so for convenience of classification and nomenclature. Further, it is understood by the artisan that, depending on the number and position of double bonds, and length and position of the branches, certain alcohols having certain required carbon atoms may have a melting point of less than the above preferred in the present invention. Such alcohols of low melting point are not intended to be included in this section. Nonlimiting examples of the high melting point alcohols are found in International Cosmetic Ingredient Dictionary, Fifth Edition, 1993, and CTFA Cosmetic Ingredient Handbook, Second Edition, 1992.

The high melting point fatty alcohols useful herein are those having from about 14 to about 30 carbon atoms, preferably from about 16 to about 22 carbon atoms. These fatty alcohols are saturated and can be straight or branched chain alcohols.

Preferred fatty alcohols include, for example, cetyl alcohol (having a melting point of about 56° C.), stearyl alcohol (having a melting point of about 58-59° C.), behenyl alcohol (having a melting point of about 71° C.), and mixtures thereof. These alcohols are known to have the above melting point. However, they often have lower melting points when supplied, since such supplied products are often mixtures of fatty alcohols having alkyl chain length distribution in which the main alkyl chain is a cetyl, stearyl or behenyl group. In the present invention, more preferred fatty alcohols are cetyl alcohol, stearyl alcohol and mixtures thereof.

Commercially available high melting point fatty alcohols useful herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan).

Together with a high melting point fatty alcohol, the compositions can further comprise a low melting fatty alcohol, for example, oleyl alcohol.

### Aqueous Carrier

The conditioning composition of the present invention comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 30% to about 95%, and more preferably from about 70% to about 90% water.

### Silicone Compound

The compositions of the present invention may, or may not, contain a silicone compound. It is believed that the silicone compound can provide smoothness and softness on dry hair. The silicone compounds herein can be used at levels by weight of the composition of preferably from about 0.1% to about 20%, more preferably from about 0.5% to about 10%, still more preferably from about 1% to about 8%.

Preferably, the silicone compounds have an average particle size of from about 1 micron to about 50 microns, in the composition.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000 mPa·s at 25° C. The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

Preferred polyalkyl siloxanes include, for example, polydimethylsiloxane, polydiethylsiloxane, and polymethylphenylsiloxane. Polydimethylsiloxane, which is also known as dimethicone, is especially preferred. These silicone compounds are available, for example, from the General Electric Company in their Viscasil® and TSF 451 series, and from Dow Corning in their Dow Corning SH200 series.

The above polyalkylsiloxanes are available, for example, as a mixture with silicone compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 100,000 mPa·s to about 30,000,000 mPa·s at 25° C., preferably from about 100,000 mPa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

Silicone compounds useful herein also include amino substituted materials. Preferred aminosilicones include, for example, those which conform to the general formula (I):

(R₁)ₐG₃₋ₐ-Si—(−OSiG₂)ₙ-(−OSiG_{b}(R₁)_{2-b})ₘ—O-SiG₃₋ₐ(R₁)ₐ

wherein G is hydrogen, phenyl, hydroxy, or C1-C8 alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C20; A⁻ is a halide ion.

Highly preferred amino silicones are those corresponding to formula (I) wherein m=0, a=1, q=3, G=methyl, n is preferably from about 1500 to about 1700, more preferably about 1600; and L is —N(CH3)2 or —NH2, more preferably —NH2. Another highly preferred amino silicones are those corresponding to formula (I) wherein m=0, a=1, q=3, G=methyl, n is preferably from about 400 to about 600, more preferably about 500; and L is —N(CH3)2 or —NH2, more preferably —NH2. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those having alkylamino substitutions as pendant groups of a silicone backbone. Highly preferred are those known as “amodimethicone”. Commercially available amodimethicones useful herein include, for example, BY16-872 available from Dow Corning. Some embodiments may include Silicone Quaternium-26.

The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; coloring agents, such as any of the FD&C or D&C dyes; perfumes; and sequestering agents, such as disodium ethylenediamine tetra-acetate; ultraviolet and infrared screening and absorbing agents such as benzophenones; and antidandruff agents such as zinc pyrithione.

### Low Melting Point Oil

The compositions may comprise one or more conditioning oils. Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils including pentaerythritol tetraisostearate, trimethylol ester oils, citrate ester oils, and glyceryl ester oils; poly α-olefin oils such as polydecenes; and mixtures thereof. Additional oils may include polyester oil or mono-, di, tri-ether or ester including triglycerides, such as caprylic capric triglyceride or vegetable oils such as coconut oil, soybean oil, rapeseed oil, cocoa butter, olive oil, palm oil, rice bran oil, and mixtures thereof.

In some embodiments, a conditioning oil may have a hydrophilic-lipophilic balance (HLB) of less than about 10. In some embodiments, the oil may be a mono, di, or tri ester or ether where the monomer units have a carbon chain of C2 to C16, preferably C4 to C10, or more preferably C6 to C8. In some embodiments, the oil may be a polyester with the hydrophobic monomer units (linear or branched) having carbon chains shorter than C16, preferably shorter than C12. Commercially available oil examples include, but are not limited to, Myritol 318 from BASF (caprylic/capric triglyceride), Plantasil Micro from BASF (dicaprylyl ether in emulsion form (Dicaprylyl Ether (and) Decyl Glucoside (and) Glyceryl Oleate)); or Citropol 1A from P2 science (Polycitronellol Acetate).

### Product Forms

The conditioning compositions of the present invention can be in the form of rinse-off products or leave-on products and can be formulated in a wide variety of product forms, including but not limited to pastes, creams, gels, emulsions, mousses, and sprays. The conditioning composition of the present invention is especially suitable for a rinse-off hair conditioner or for a no-rinse hair conditioner.

### Method of Use

The conditioning composition of the present invention is preferably used for a method of conditioning hair, the method comprising following steps:

(i) after shampooing hair, applying to the hair an effective amount of the conditioning composition for conditioning the hair; and
(ii) optionally, then rinsing the hair.

Effective amount herein is, for example, from about 0.1 ml to about 2 ml per 10 g of hair, preferably from about 0.2 ml to about 1.5 ml per 10 g of hair.

The conditioning composition of the present invention provides improved conditioning benefits, especially improved wet conditioning benefits after rinsing and improved dry conditioning, while maintaining wet conditioning benefit before rinsing. The conditioning composition of the present invention may also provide improved product appearance to consumer. Thus, a reduced dosage of the conditioning composition of the present invention may provide the same level of conditioning benefits as those of a full dosage of conventional conditioner compositions. Such reduced dosage herein is, for example, from about 0.3 ml to about 0.7 ml per 10 g of hair.

### Method of Manufacturing

The present invention is also directed to a method of manufacturing a hair conditioning composition as follows:

A method of making the hair conditioner composition comprising:
a material comprising a first material and a second material;
wherein the first material has at least two amine groups, has an A Log P at a pH of 6 of at less than about −2, and has a hydrophobic surface area at pH 6 of at least about 160 Å² (Angstrom)²;

wherein the second material comprises at least one C12 to C22 carbon chain and has an A Log P at pH 6 of at least about 3.2 b. a high melting point fatty alcohol; and

c. an aqueous carrier;

said method comprising the following steps:
a) Add water that is at a temperature higher than the temperature of the melting point of the second material, the fatty alcohol, and mixture of them (about 80° C.-90° C.);
b) Add the first material in hot water (about 80° C.-90° C.) before adding of fatty alcohol and the second material;
c) Prepare a homogeneous premix by combining the second material and a fatty alcohol at a temperature that is higher than either of their individual melting points and add it into hot water (about 80° C.-90° C.);
d) Cool the mixture below the phase transition temperature to form a gel structure.

### Method of Preparation

Water is prepared at from about 80° C. to about 90° C. The first material is added in the prepared hot water. Fatty alcohols and the second material then are added in the prepared hot solution and prepared dispersed homogenous mixture at about 85° C. After, the mixture is cooled down to below 40° C. with agitation. A gel structure (no phase separation) is formed. If included, silicone compounds, perfumes, preservatives are added to the gel structure with agitation after the composition is cooled down to room temperature.

The present invention is also directed to a second method of manufacturing a hair conditioning composition as follows:
a) Add water that is at a temperature higher than the temperature of the melting point of the second material, the fatty alcohol, and mixture of them;
b) Add fatty alcohols and wait until dissolved and dispersed homogenously;
c) Add the second material and wait until dissolved and dispersed homogenously;
d) Add the first material and wait until dissolved and dispersed homogenously;
e) Cool the mixture below the phase transition temperature to form a gel network matrix; and
f) Add the remaining ingredients either before or after the gel network matrix formation, while maintaining pH of at least about 4.5.

An alternative method may be to prepare a homogeneous premix by combining a second material and a fatty alcohol at a temperature that is higher than either of their individual melting points and add it into hot water (about 80-90° C.) instead of steps of b) and c). Another alternative method may be to add the first and second material in hot water (about 80-90° C.) before adding of fatty alcohol and the second material.

A further alternative method of making a hair conditioner composition may comprise the following steps:

a) making a solid shape material by combining the first material, a fatty alcohol, and the second material; and then b) combining a) in an aqueous chassis.

The method may further comprise the steps of adding additional ingredients such as silicone or oil compounds, perfumes, preservatives, esthetics if included, to gel structure. The inventive conditioning compositions of the present invention can be prepared by any conventional method well known in the art.

The pH of the finished product and of the composition during the making process after the cooling down step may be at least 4.5.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

Table 1 shows the results for gel formation for non-basic amino acid first material with C12 Fatty Acid second material (Ex. 1, 2) and gel formation for basic amino acid first material with non-fatty acids C18 sugar ester second material (Ex. 3). See example, Guanine with C12-18 Fatty Acid (C. Ex. 1 and 2) and Hydroxyethylurea with C18 Fatty Acid (C. Ex. 3) exhibits phase separation, which indicates that the gel structure/matrix cannot be formed.

TABLE 1

| | Ex. 1 | Ex. 2 | Ex. 3 | C. Ex. 1 | C. Ex. 2 | C. Ex. 3 |
|---|---|---|---|---|---|---|
| Description | Inventive | Inventive | Inventive | C. Ex. 1: | C. Ex. 2: | C. Ex. 3: |
| | Example 1: | Example 2: | Example 3: | Guanine: | Guanine: | Hydroxyethylurea: |
| | Canavanine: | DMAPA: | Histidine: | Lauric | Oleic | Oleic Acid |
| | Lauric | Lauric | Sucrose Di | Acid | Acid | |
| | Acid | Acid | Stearate | | | |
| First | −4.8 | −3.01 | −2.58 | −0.66 | −0.66 | −1.03 |
| Material_ALogP | | | | | | |
| (ALogP < −2) | | | | | | |
| First | 179 | 238 | 181 | 153 | 153 | 79 |
| Material_HSA | | | | | | |
| (HSA > 160) | | | | | | |
| Second | 3.48 | 3.48 | 11.47 | 3.48 | 6.48 | 6.48 |
| Material_ALogP | | | | | | |
| (ALogP > 3.2) | | | | | | |
| Canavanine | 0.22 | — | — | — | — | — |
| DMAPA | — | 0.22 | | — | — | — |
| Histidine | | | 0.22 | | | |
| Guanine | — | — | — | 0.22 | 0.22 | |
| Hydroxyethyl Urea* | — | — | — | — | — | 0.44 |
| Lauric Acid | 2.17 | 2.17 | — | 2.17 | — | — |
| Oleic Acid | — | — | — | — | 2.17 | 2.17 |
| Sucrose Di Stearate | — | — | 2.17 | — | — | — |
| Cetyl Alcohol | 1.75 | 1.75 | 1.75 | 1.75 | 1.75 | 1.75 |
| Stearyl Alcohol | 3.02 | 3.02 | 3.02 | 3.02 | 3.02 | 3.02 |
| Water | Q.S, | Q.S, | Q.S, | Q.S, | Q.S, | Q.S, |
| pH | 6.2 [symbol] | 6.7 | 7.2 | NA | NA | NA |
| Rheology Shear Stress | 213 [symbol] | 526. [symbol] | 494. [symbol] | NA | NA | NA |
| at 950 S−1 (Pa) | | | | | | |
| Product Stability | Stable | Stable | Stable | Phase | Phase | Phase |
| | | | | Separation | Separation | Separation |
| Conditioning | Acceptable | Acceptable | Acceptable | — | — | — |

*It has about 50/50 active/water level distribution

Table 2: shows the results for gel formation for basic amino acid first material with non-fatty acids C18 and C12 sugar ester and C18 Glyceryl ester (Ex. 4-7). In contrast, Hydroxyethylurea with C18 Glyceryl ester (C. Ex. 4) and Guanine with C12 sugar ester (C. Ex. 5) exhibits phase separation, which indicates that the gel structure/matrix cannot be formed.

TABLE 2

| | Ex. 4 | Ex. 5 | Ex. 6 | Ex. 7 | C. Ex. 4 | C. Ex. 5 |
|---|---|---|---|---|---|---|
| First | −3.93 | −3.93 | −2.58 | −2.58 | −1.03 | −0.66 |
| Material_ALogP | | | | | | |
| (ALogP < −2) | | | | | | |
| First | 198 | 198 | 181 | 181 | 79 | 153 |
| Material_HSA | | | | | | |
| (HSA > 160) | | | | | | |
| Second | 11.47 | 5.33 | 5.31 | 5.33 | 5.33 | 5.31 |
| Material_ALogP | | | | | | |
| (ALogP > 3.2) | | | | | | |
| Description | Inventive | Inventive | Inventive | Inventive | C. Ex. 4: | C. Ex. 5: |
| | Example 4: | Example 5: | Example 6: | Example 7: | Hydroxyethylurea: | Guanine: |
| | Arginine: | Arginine: | Histidine: | Histidine: | Glyceryl Oleate | Sucrose Di |
| | Sucrose Di | Glyceryl | Sucrose Di | Glyceryl | | Laurate |
| | Stearate | Oleate | Laurate | Oleate | | |
| Hydroxyethyl Urea* | — | — | — | — | 0.44 | — |
| Histidine | — | — | 0.22 | 0.22 | — | — |
| Guanine | — | — | — | — | — | 0.22 |
| L-Arginine | 0.22 | 0.22 | — | — | — | — |
| Sucrose Di Laurate | — | — | 2.17 | — | — | 2.17 |
| Sucrose Di Stearate | 2.17 | — | — | 2.17 | — | — |
| Glyceryl Oleate | — | 2.17 | — | 2.17 | 2.17 | — |
| Cetyl Alcohol | 1.75 | 1.75 | 1.75 | 1.75 | 1.75 | 1.75 |
| Stearyl Alcohol | 3.02 | 3.02 | 3.02 | 3.02 | 3.02 | 3.02 |
| Water | Q.S, | Q.S, | Q.S, | Q.S, | Q.S, | Q.S, |
| pH | 9.8 | 9.6 | 7.6 | 7.3 | NA | NA |
| Rheology Shear Stress | 332 [symbol] | 583. [symbol] | 87 | 494. [symbol] | NA | NA |
| at 950 S−1 (Pa) | | | | | | |
| Product Stability | Stable | Stable | Stable | Stable | Phase | Phase |
| | | | | | Separation | Separation |
| Conditioning | Acceptable | Acceptable | Acceptable | Acceptable | NA | NA |

*It has about 50/50 active/water level distribution

Table 3: Shows the results for gel formation for with non-fatty acids C18 sugar ester and C18 Glyceryl ester as second material and basic amino acid (Lysine) as first material (Ex. 8 and 9). In contrast, Dodecyl glucoside as second material with non-basic amino acid and basic amino acid first material exhibits phase separation (C. Ex. 6 and 7) and Hydroxyethylurea as first material with C18 sugar ester tail (C. Ex. 8) and Guanine head with Dodecyl glucoside tail (C. Ex. 9) exhibits phase separation, which indicates that the gel structure/matrix cannot be formed.

TABLE 3

| | Ex. 8 | Ex. 9 | C. Ex. 6 | C. Ex. 7 | C. Ex. 8 | C. Ex. 9 |
|---|---|---|---|---|---|---|
| First | −4.7 | −4.7 | −3.01 | −3.93 | −1.03 | −0.66 |
| Material_ALogP | | | | | | |
| (ALogP < −2) | | | | | | |
| First | 175 | 175 | 238 | 198 | 79 | 153 |
| Material_HSA | | | | | | |
| (HAS > 160) | | | | | | |
| Second | 11.47 | 5.33 | 2.87 | 2.87 | 11.47 | 2.87 |
| Material_ALogP | | | | | | |
| (ALogP > 3.2) | | | | | | |
| Description | Inventive | Inventive | C. Ex. 6 | C. Ex. 7: | C. Ex. 8: | C. Ex. 9: |
| | Example 8: | Example 9: | DMAPA: | Arginine: | HydroxyethylUrea: | Guanine: |
| | Lysine: | Lysine: | Dodecyl | Dodecyl | Sucrose Di | Dodecyl |
| | Sucrose Di | Glyceryl | glucoside | glucoside | Stearate | glucoside |
| | Stearate | Oleate | | | | |
| Hydroxyethyl Urea* | — | — | — | — | 0.44 | — |
| Lysine | 0.22 | 0.22 | — | — | — | — |
| DMAPA | — | — | 0.22 | — | — | — |
| Guanine | — | — | — | — | — | 0.22 |
| L-Arginine | — | — | — | 0.22 | — | — |
| Dodecyl glucoside | — | — | 2.17 | 2.17 | — | 2.17 |
| Glyceryl Oleate | — | 2.17 | — | — | — | — |
| Sucrose Di Stearate | 2.17 | — | — | — | 2.17 | — |
| Cetyl Alcohol | 1.75 | 1.75 | 1.75 | 1.75 | 1.75 | 1.75 |
| Stearyl Alcohol | 3.02 | 3.02 | 3.02 | 3.02 | 3.02 | 3.02 |
| Water | Q.S, | Q.S, | Q.S, | Q.S, | Q.S, | Q.S, |
| pH | 9.8 | 8.9 | NA | NA | NA | NA |
| Rheology Shear Stress | 253. [symbol] | 272. [symbol] | NA | NA | NA | NA |
| at 950 S−1 (Pa) | | | | | | |
| Product Stability | Stable | Stable | Phase | Phase | Phase | Phase |
| | | | Separation | Separation | Separation | Separation |
| Conditioning | Acceptable | Acceptable | NA | NA | NA | NA |

*It has about 50/50 active/water level distribution

### Test Methods

1. Product Stability Visual Assessment

Stability is the visual assessment to ensure the product is consistently stable over a specific period. The composition of product was placed in three different condition −5° C., 25° C. and 40° C. The assessment will be taken at different interval:

1) Stable as made
2) Stable after one week

The description test for Product Stability: If the visual assessment of the product is stable at different interval, it will be stated as Stable. If the visual assessment of the product exhibit phase separation at either of the interval, it will be stated as Phase Separation.

2. General Conditioning Sensory Method

This is expert sensory panel test method uses three highly expert sensory panel to evaluate specific attribute during dry stage hair treatment. The treatment protocol for the hair treatment is stated as follow:
a. Rinse 20 g of hair switches with water and squeeze water out from top to bottom once.
b. Apply 1 ml conditioner on front and apply 1 ml conditioner on back.
c. Lather the product 30 strokes for 30 seconds on hair switch.
d. Rinse for 15 seconds front and 15 seconds back. and squeeze water out from top to bottom once.
a. Leave overnight to dry

The sensory attribute evaluated during this method is notated as “Conditioning” in the Example tables. Panelist evaluated wet stage for wet spreadability and slip, and evaluated dry smoothness during dry stage, and was asked to find it either “Acceptable” or “Not Acceptable” (as conditioning feel).

3. Rheology Method

Rheology is used to evaluate and characterise product samples. Shear Stress at 950 s⁻¹ via flow curve: This is the method to ramp up shear rate logarithmically from 0.1 to 1000 s⁻¹ in 1 min using a cone & plate geometry, and to read the shear stress value σ (Pa) at shear rate 950 s⁻¹.

The acceptable rheology range for shear stress is from 5 Pa until 1500 Pa.

4. A Log P:

It is Octanol/Water partition coefficient which talks about the hydrophilicity or hydrophobicity of the molecule. A log P″ as used herein, is an identification of the octanol-water partition coefficient of an active. Ghose and Crippen used this atom-based method to calculate the octanol-water partition coefficient (log P), and the molar refractivity (MR) for incoming molecules. Log P provides a measure of the hydrophobicity of the molecule, while MR contains information about molecular volume and polarizability. A log P is calculated herein using Pipeline Pilot software (Biovia™) ver 9.2.

5. Hydrophobic Surface Area (HSA):

HSA describe the surface area of the no-polar atoms on the molecules, which helps to understand the hydrophobic part of the molecule.

Computational Procedure: Commercial Pipeline Pilot Software (from Dassault System) was used to calculate all the properties except for HLB values, for which we used Marvin Sketch software.

### EXAMPLES/COMBINATIONS

A. A hair conditioner composition comprising:
   a first material and a second material;
      wherein the first material comprises at least two amine groups, has an A Log P at pH 6 of at less than about −2, and has a hydrophobic surface area at pH 6 of at least about 160 Å² (Angstrom)²; and
      wherein the second material comprises at least one C12 to C22 carbon chain and has an A Log P at pH 6 of at least about 3.2.
B. The composition of paragraph A, wherein the second material is selected from the group consisting of a fatty acid, sugar esters, glycerol esters and ethers with at least one double bond, and sulphates.
C. The composition of any one of paragraphs A or B, wherein the composition further comprises a fatty alcohol.
D. The composition of any one of paragraphs A to C, wherein the composition further comprises an aqueous carrier.
E. The composition of any one of paragraphs A to D, wherein the composition has a gel structure.
F. The composition of any one of paragraphs A to E, wherein the composition has a pH from about 6 to about 11.
G. The composition of any one of paragraphs A to F, wherein the composition has an HLB range from 8 to 38.
H. The composition of any one of paragraphs A to G, wherein the second material is selected from the group consisting of sucrose distearate, sucrose dipalmitate, sucrose dilaurate, glyceryl palmitoleate, glyceryl monooleate, glyceryl lionleate, and glyceryl linolenate.
I. The composition of any one of paragraphs A to H, wherein the first material is selected from the group consisting of canavanine, lysine, ornithine, arginine, citrulline, methylarginine, laminine, ethanimidoylornithine, n-dimethylarginine, norleucine, argininamide, DMAPA, propyl arginine, histidine, and 4-aminophenylalanine.
J. The composition of any one of paragraphs A to I, wherein the first material is canavanine or ornithine.
K. The composition of any one of paragraphs A to J, wherein the composition further comprises a conditioning oil.
L. The composition of paragraph K, wherein the conditioning oil is a non-silicone.
M. The composition of paragraph K, wherein the conditioning oil is in preformed emulsion form, with a particle size at most about 500 nm.
N. The composition of paragraph K, wherein the conditioning oil has an HLB of less than about 10.
O. The composition of any one of paragraphs A to N, wherein the first material is arginine and the second material is sucrose distearate or glyceryl monooleate.
P. The composition of any one of paragraphs A to 0, wherein the first material is canavanine and the second material is lauric acid.
Q. The composition of any one of paragraphs A to P, wherein the first material is histidine and the second material is selected from the group consisting of glyceryl monooleate, sucrose distearate, and sucrose dilaurate.
R. The composition of any one of paragraphs A to Q, wherein the first material is lysine and the second material is glyceryl monooleate or sucrose distearate.
S. The composition of any one of paragraphs A to R, wherein the first material is DMAPA and the second material is lauric acid.
T. The composition of any one of paragraphs A to S, wherein the hair conditioner composition comprises from about 0.01% to about 15% of the first material, by weight of the hair conditioner composition; and wherein the hair conditioner composition comprises from about 0.01% to about 15% of the second material, by weight of the hair conditioner composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair conditioner composition comprising:
a first material and a second material;
wherein the first material comprises at least two amine groups, has an A Log P at pH 6 of less than about −2, and has a hydrophobic surface area at pH 6 of at least about 160 Å² (Angstrom)²; and
wherein the second material comprises at least one C12 to C22 carbon chain and has an A Log P at pH 6 of at least about 3.2.

2. The hair conditioner composition of claim 1, wherein the second material is selected from the group consisting of a fatty acid, sugar esters, glycerol esters and ethers with at least one double bond, and sulphates.

3. The hair conditioner composition of claim 1, wherein the composition further comprises a fatty alcohol.

4. The hair conditioner composition of claim 1, wherein the composition further comprises an aqueous carrier.

5. The hair conditioner composition of claim 1, wherein the composition has a gel structure.

6. The hair conditioner composition of claim 1, wherein the composition has a pH from about 5 to about 11.

7. The hair conditioner composition of claim 1, wherein the composition has an HLB range from 8 to 38.

8. The hair conditioner composition of claim 1, wherein the second material is selected from the group consisting of sucrose distearate, sucrose dipalmitate, sucrose dilaurate, glyceryl palmitoleate, glyceryl monooleate, glyceryl lionleate, and glyceryl linolenate.

9. The hair conditioner composition of claim 1, wherein the first material is selected from the group consisting of canavanine, lysine, ornithine, arginine, citrulline, methylarginine, laminine, ethanimidoylornithine, n-dimethylarginine, norleucine, argininamide, DMAPA, propyl arginine, histidine, and 4-aminophenylalanine.

10. The hair conditioner composition of claim 1, wherein the first material is canavanine or ornithine.

11. The hair conditioner composition of claim 1, wherein the composition further comprises a conditioning oil.

12. The hair conditioner composition of claim 11, wherein the conditioning oil is a non-silicone.

13. The hair conditioner composition of claim 11, wherein the conditioning oil is in preformed emulsion form, with a particle size at most about 500 nm.

14. The hair conditioner composition of claim 11, wherein the conditioning oil has an HLB of less than about 10.

15. The hair conditioner composition of claim 1, wherein the first material is arginine and the second material is sucrose distearate or glyceryl monooleate.

16. The hair conditioner composition of claim 1, wherein the first material is canavanine and the second material is lauric acid.

17. The hair conditioner composition of claim 1, wherein the first material is histidine and the second material is selected from the group consisting of glyceryl monooleate, sucrose distearate, and sucrose dilaurate.

18. The hair conditioner composition of claim 1, wherein the first material is lysine and the second material is glyceryl monooleate or sucrose distearate.

19. The hair conditioner composition of claim 1, wherein the first material is DMAPA and the second material is lauric acid.

20. The hair conditioner composition of claim 1, wherein the hair conditioner composition comprises from about 0.01% to about 15% of the first material, by weight of the hair conditioner composition; and wherein the hair conditioner composition comprises from about 0.01% to about 15% of the second material, by weight of the hair conditioner composition.
